(19) United States
(12) Patent Application Publication
(10) Pub. No.: US 2004/0241880 A1
(43) Pub. Date: Dec. 2, 2004

(54) Ligand array assays having reduced fluorescent dye degradation and compositions for practicing the same

(76) Inventors: Eric M. Leproust, San Jose, CA (US); Winny W. Ke, San Jose, CA (US); Daniel B. Roitman, Menlo Park, CA (US); Bill J. Peck, Mountain View, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/452,801

(22) Filed: May 30, 2003

(51) Int. Cl. G01N 33/543

(52) U.S. Cl. 436/518

(57) ABSTRACT

Ligand array assays that exhibit reduced dye degradation and compositions for use in practicing the same are provided. A feature of the subject methods is that they include a label degradation inhibitor deposition step. In this degradation inhibitor deposition step, the surface of a sample exposed array is contacted with a low surface tension fluid, e.g., acetonitrile, that includes a fluorescent dye degradation inhibitor. Also provided are kits for use in practicing the subject methods. The subject methods-and kits find use in a variety of ligand array based applications, including genomic and proteomic applications.

### FIELD OF THE INVENTION

The present invention relates to ligand, and particularly, biopolymeric arrays.

### BACKGROUND OF THE INVENTION

Array assays between surface bound binding agents or probes (i.e., ligands) and target molecules in solution may be used to detect the presence of particular analytes in the solution. The surface-bound probes may be nucleic acids (e.g., oligonucleotides, polynucleotides), peptides (e.g., polypeptides, proteins, antibodies) or other molecules capable of binding with target biomolecules in the solution (e.g., nucleic acids, proteins, etc.). Such binding interactions are the basis for many of the methods and devices used in a variety of different fields, e.g., genomics (in sequencing by hybridization, SNP detection, differential gene expression analysis, identification of novel genes, gene mapping, finger printing, etc.) and proteomics.

One typical array assay method involves biopolymeric probes immobilized in discrete locations on a surface of a substrate (collectively referred to herein as an “array”) such as a glass substrate or the like. A solution containing target molecules (“targets”) that bind with the attached probes is placed in contact with the bound probes under conditions sufficient to promote binding of targets in the solution to the complementary probes on the substrate to form a binding complex that is bound to the surface of the substrate. The binding by target molecules to probe features or spots on the substrate produces a pattern, i.e., a binding complex pattern, on the surface of the substrate, which pattern is then detected. This detection of binding complexes provides desired information about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

In many array-based assays, fluorescent labels are employed to label target molecules that are bound to surface immobilized probes of the array. Representative fluorescent labels that find use in various array protocols currently practiced in the art include xanthene dyes, e.g. fluorescein and rhodamine dyes, such as fluorescein isothiocyanate (FITC), 6-carboxyfluorescein (commonly known by the abbreviations FAM and F),6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes; quinoline dyes; and benzopyrylium-based fluorescent dyes.

While fluorescent labels are used frequently in array-based applications, it is well recognized in the art that the fluorescent dyes are susceptible to degradation and loss of fluorescent activity due to their relatively elevated chemical reactivity. Such degradation can have a significant adverse impact on the results obtained from a given assay. For example, in a two color assay where two distinguishably fluorescent labeled samples are compared, one of the fluorescent dyes may be degraded to an extent greater than the other, leading to significant inaccuracies in the observed results of the assay.

As such, there is a need in the art to develop an effective way to substantially reduce, if not eliminate, fluorescent label degradation in array-based assays. The present invention satisfies this need.

Relevant Literature

Published U.S. patent application Nos. 20030003496A1 and 20020006622A1, as well as published PCT application WO 01/94630. Also see U.S. Pat. No. 6,428,748.

### SUMMARY OF THE INVENTION

Ligand array assays that exhibit reduced dye degradation and compositions for use in practicing the same are provided. A feature of the subject methods is that they include a label degradation inhibitor deposition step. In this degradation inhibitor deposition step, the surface of a sample exposed array is contacted with a low surface tension fluid, e.g., acetonitrile, that includes a fluorescent dye degradation inhibitor. Also provided are kits for use in practicing the subject methods. The subject methods and kits find use in a variety of ligand array based applications, including genomic and proteomic applications.

### BRIEF DESCRIPTION OF THE FIGURES

FIG. 1 shows an exemplary substrate carrying an array, such as may be used in the devices of the subject invention.

FIG. 2 shows an enlarged view of a portion of FIG. 1 showing spots or features.

FIG. 3 is an enlarged view of a portion of the substrate of FIG. 2.

FIGS. 4A and 4B show the morphologies of representative features observed in an experiment described in the Experimental Section, below.

FIGS. 5A and 5B show that the degradation of Cy5 is a pseudo first order reaction correlated with the concentration of ozone.

FIG. 6 provides a graphical comparison of results obtained with or without using a fluorescent dye degradation inhibitor according to the present invention.

FIGS. 7A and B provide the results of an array assay performed according to the subject invention and a control.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “biomolecule” means any organic or biochemical molecule, group or species of interest that may be formed in an array on a substrate surface. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

A “biopolymer” is a polymeric biomolecule of one or more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups.

A “biomonomer” references a single unit, which can be linked with the same or other biomonomers to form a biopolymer (e.g., a single amino acid or nucleotide with two linking groups, one or both of which may have removable protecting groups).

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

With respect to methods in which pre-made probes are immobilized on a substrate surface, immobilization of the probe to a suitable substrate may be performed using conventional techniques. See, e.g., Letsinger et al. (1975) Nucl. Acids Res. 2:773-786; Pease, A. C. et al., Proc. Nat. Acad. Sci. USA, 1994, 91:5022-5026. The surface of a substrate-may be treated with an organosilane coupling agent to functionalize the surface. One exemplary organosilane coupling agent is represented by the formula RₙSiY₍₄₋ₙ₎ wherein: Y represents a hydrolyzable group, e.g., alkoxy, typically lower alkoxy, acyloxy, lower acyloxy, amine, halogen, typically chlorine, or the like; R represents a nonhydrolyzable organic radical that possesses a functionality which enables the coupling agent to bond with organic resins and polymers; and n is 1, 2 or 3, usually 1. One example of such an organosilane coupling agent is 3-glycidoxypropyltrimethoxysilane (“GOPS”), the coupling chemistry of which is well-known in the art. See, e.g., Arkins, “Silane Coupling Agent Chemistry,” Petrarch Systems Register and Review, Eds. Anderson et al. (1987). Other examples of organosilane coupling agents are (γ-aminopropyl)triethoxysilane and (γ-aminopropyl)trimethoxysilane. Still other suitable coupling agents are well known to those skilled in the art. Thus, once the organosilane coupling agent has been covalently attached to the support surface, the agent may be derivatized, if necessary, to provide for surface functional groups. In this manner, support surfaces may be coated with functional groups such as amino, carboxyl, hydroxyl, epoxy, aldehyde and the like.

Use of the above-functionalized coatings on a solid support provides a means for selectively attaching probes to the support. For example, an oligonucleotide probe formed as described above may be provided with a 5′-terminal amino group that can be reacted to form an amide bond with a surface carboxyl using carbodiimide coupling agents. 5′ attachment of the oligonucleotide may also be effected using surface hydroxyl groups activated with cyanogen bromide to react with 5′-terminal amino groups. 3′-terminal attachment of an oligonucleotide probe may be effected using, for example, a hydroxyl or protected hydroxyl surface functionality.

In situ prepared ligand arrays, e.g., nucleic acid arrays, may be characterized by having surface properties of the substrate that differ significantly between the feature and inter-feature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water. “Contact angle” of a liquid with a surface is the acute angle measured between the edge of a drop of liquid on that surface and the surface. Contact angle measurements are well known and can be obtained by various instruments such as an FTA200 available from First Ten Angstroms, Portsmouth, Va., U.S.A. Surfaces which are more hydrophobic (which have a lower surface energy) will have higher contact angles with water or aqueous liquids than surfaces which are less hydrophobic (and therefore a higher surface energy) (for example, a hydrophobic surface may have a water drop contact angle of more than 50 degrees, or even more than 90 degrees). The contact angle of an array (sometimes referenced as the “average contact angle” or “effective contact angle”) is the average contact angle of the features of that array and the inter-feature areas. Contact angles are measured with water unless otherwise indicated.

In certain embodiments, high surface energy regions, e.g., features, may have contact angles that are less than 45 degrees, less than 20 degrees (or less than 15, 10, or 5 degrees), while low surface energy, e.g., inter-feature, areas may have contact angles greater than 80 degrees (or even greater than 90, 95, 100,105, 110, 115, 120 or 130 degrees).

Also, instead of drop deposition methods, light directed fabrication methods may be used, as are known in the art. Inter-feature areas need not be present particularly when the arrays are made by light directed synthesis protocols.

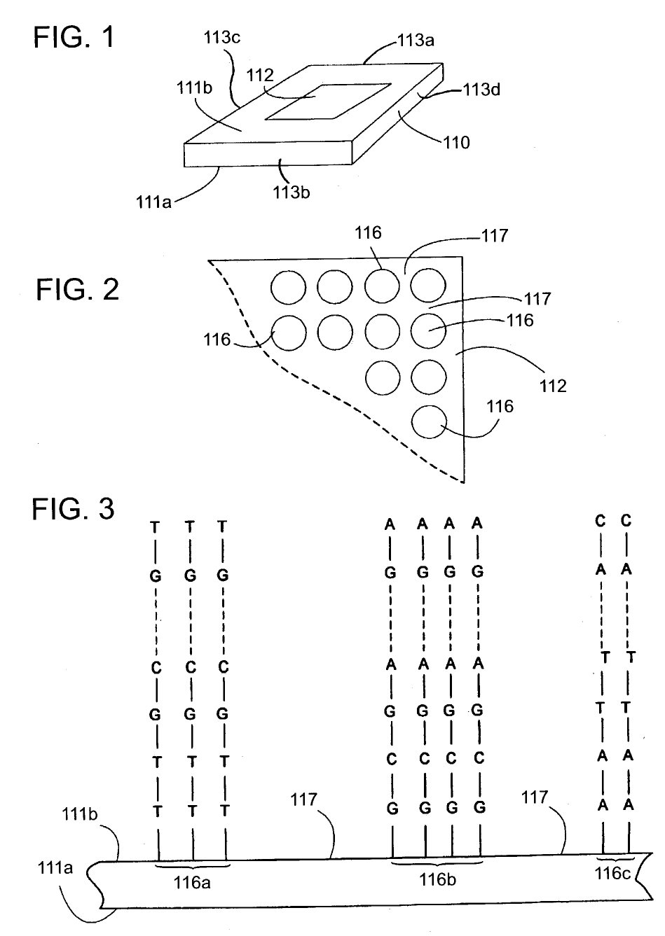

An exemplary array is shown in FIGS. 1 to 3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage. “Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure, e.g., a bottom surface or a cover that does not readily bend without breakage, i.e., the structure is not flexible.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equilvalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “sample” as used herein relates to a material or mixture of materials, typically, although not necessarily, in fluid form, containing one or more components of interest.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DESCRIPTION OF THE SPECIFIC EMBODIMENTS

Ligand array assays that exhibit reduced dye degradation and compositions for use in practicing the same are provided. A feature of the subject methods is that they include a label degradation inhibitor deposition step. In this degradation inhibitor deposition step, the surface of a sample exposed array is contacted with a low surface tension fluid, e.g., acetonitrile, that includes a fluorescent dye degradation inhibitor. Also provided are kits for use in practicing the subject methods. The subject methods and kits find use in a variety of ligand array based applications, including genomic and proteomic applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

Introduction

As summarized above, the subject invention provides methods and kits for performing array-based assays, i.e., array-binding assays. The subject invention can be used with a number of different types of arrays in which a plurality of distinct polymeric binding agents (i.e., of differing sequence) are stably associated with at least one surface of a substrate or solid support. The polymeric binding agents may vary widely, however polymeric binding agents of particular interest include peptides, proteins, nucleic acids, polysaccharides, synthetic mimetics of such biopolymeric binding agents, etc. In many embodiments of interest, the biopolymeric arrays are arrays of nucleic acids, including oligonucleotides, polynucleotides, cDNAs, mRNAs, synthetic mimetics thereof, and the like.

While the subject methods and devices find use in array hybridization assays, the subject devices also find use in any suitable binding assay in which members of a specific binding pair, e.g., a ligand and receptor, interact. That is, any of a number of different binding assays may be performed with the subject methods, where a first member of a binding pair, typically referred to herein as the ligand, is stably associated with the surface of a substrate and a second member of a binding pair, which may be referred to as the receptor for the ligand, is free in a sample, where the binding members may be: antibodies and antigens, complementary nucleic acids, and the like. For ease of description only, the subject methods and devices described below will be described primarily in reference to hybridization assays, where such examples are not intended to limit the scope of the invention. It will be appreciated by those of skill in the art that the subject devices and methods may be employed for use with other binding assays as well, such as immunoassays, proteomic assays, etc.

In further describing the subject invention, the subject methods are described first in greater detail, followed by a review of representative applications in which the subject methods find use, as well as a review of representative systems and kits that find use in practicing the subject methods.

Methods

As summarized above, methods are provided for performing an array-based assay such as a hybridization assay or any other analogous binding interaction assay. A feature of the present methods is that they include a degradation inhibitor deposition step, in which the surface of a sample exposed array is contacted with a low surface tension fluid, e.g., acetonitrile, that includes a fluorescent dye degradation inhibitor.

In practicing the subject methods, the first step is typically to fluorescently label a sample, which in many embodiments is at least suspected to have (if not known to include) an analyte of interest. By fluorescently label a sample is meant a process that fluorescently labels any analyte present in the sample, so that the analyte can be detected during the array assay using fluorescent detection protocols. A sample may be fluorescently labeled by a number of different protocols, where the selection of a given protocol depends, at least in part, on the specific nature of the array-based assay that is being performed.

For example, in nucleic acid array-based assays, a sample may be fluorescently labeled by enzymatically generating fluorescently labeled nucleic acid targets using fluorescently labeled nucleotide precursors, as is well known in the art. In yet other approaches, nucleotides labeled with a first reactive functionality are employed in the nucleic acid target generation step, where the resultant targets are then fluorescently labeled with functionalized fluorescent molecules that react with the functionalities present on the targets. In an analogous labeling approach where proteinaceous targets are the analytes, the proteinaceous targets may be labeled with functionalized fluorescent molecules that react with functionalities present on the targets. The above summarized approaches are merely representative of the variety of different fluorescent labeling protocols that are known and readily practiced by those of skill in the art, and may be employed in the sample fluorescent labeling step of the subject methods.

The fluorescent label employed in the fluorescent labeling step may vary, where a number of different types of fluorescent labels are known to those of skill in the art. Representative fluorescent labels that find use in various array protocols currently practiced in the art include xanthene dyes, e.g. fluorescein and rhodamine dyes, such as fluorescein isothiocyanate (FITC), 6-carboxyfluorescein (commonly known by the abbreviations FAM and F),6-carboxy-2′,4′,7′,4 ,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, Cy7, etc; BODIPY dyes; quinoline dyes; and Benzopyrylium-based fluorescent dyes.

Cyanine and related dyes, such as merocyanine, styryl and oxonol dyes, are particularly strongly light-absorbing and highly luminescent, see, e.g., U.S. Pat. Nos. 4,337,063; 4,404,289; 6,048,982. In one embodiment, Cy3 and Cy5 are used together; both are fluorescent cyanine dyes produced by Amersham Life Sciences (Arlington Heights, Ill.). They can be incorporated into “target” nucleic acid by transcription (e.g., by random-primer labeling using Klenow polymerase, or “nick translation,” or, amplification, or equivalent) of samples of genomic DNA, wherein the reaction incorporates Cy3-or Cy5-dCTP conjugates mixed with unlabeled dCTP. According to manufacturer's instructions, if generating labeled target by PCR, a mixture of 33% modified to 66% unmodified dCTP gives maximal incorporation of label; when modified dCTP made up 50% or greater, the PCR reaction was inhibited. Cy5 is typically excited by the 633 nm line of HeNe laser, and emission is collected at 680 nm. See also, e.g., Bartosiewicz (2000) Archives of Biochem. Biophysics 376:66-73; Schena (1996) Proc. Natl. Acad. Sci. USA 93:10614-10619; Pinkel (1998) Nature Genetics 20:207-211; Pollack (1999) Nature Genetics 23:41-46.

Following fluorescent labeling of the sample, as described above, the fluorescently labeled sample is then contacted with an array of binding agents, i.e., a ligand array, where the array includes a binding agent (ligand) specific for the analyte(s) of interest. Contact occurs under conditions sufficient for the analyte (if present) to specifically bind to its respective binding pair member that is present on the array. Thus, if the analyte of interest is present in the sample, it specifically binds to the array at the site of its complementary binding member and a ligand/analyte complex, e.g., probe/target duplex nucleic acid, is formed on the array surface. Depending on the nature of the analyte(s), the array may vary greatly, where representative arrays are reviewed in the Definitions section, above. Of particular interest are nucleic acid arrays, where in situ prepared nucleic acid array are employed in many embodiments of the subject invention.

To contact the sample with the array, the array and sample are brought together in a manner sufficient so that the sample contacts the surface immobilized ligands of the array. As such, the array may be placed on top of the sample, the sample may be placed onto, e.g., deposited onto, the array surface, the array may be immersed in the sample, etc.

Following contact of the array and the sample, the resultant sample contacted or exposed array is then maintained under conditions sufficient and for a sufficient period of time for any binding complexes between members of specific binding pairs to occur. In many embodiments, the duration of this step is at least about 10 min long, often at least about 20 min long, and may be as long as 30 min or longer, but often does not exceed about 72 hours. The sample/array structure is typically maintained at a temperature ranging from about 40 to about 80, such as from about 40 to 70° C. Where desired, the sample may be agitated to ensure contact of the sample with the array.

In the case of hybridization assays, the fluorescently labeled sample is contacted with the array under stringent hybridization conditions, whereby complexes are formed between target nucleic acids that are complementary to probe sequences attached to the array surface, i.e., duplex nucleic acids are formed on the surface of the substrate by the interaction of the probe nucleic acid and its complement target nucleic acid present in the sample. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5× Denhardt's solution, 10% dextran sulfate, followed by washing the filters in 0.1×SSC at about 65° C. Hybridization involving nucleic acids generally takes from about 30 minutes to about 24 hours, but may vary as required. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

Once the incubation step is complete, the array may be washed one or more times to remove any unbound and non-specifically bound sample from the substrate. In certain embodiments, at least two wash cycles are used. The one or more wash steps generally employ an aqueous wash fluid, where the aqueous wash fluid may include one or more washing agents. Washing agents used in array assays are known in the art and, of course, may vary depending on the particular binding pair used in the particular assay. For example, in those embodiments employing nucleic acid hybridization, washing agents of interest include, but are not limited to, salt solutions such as sodium, sodium phosphate and sodium, sodium chloride and the like as is known in the art, at different concentrations and may include some surfactant as well. [As mentioned above, a feature of the subject invention is that the methods include a label degradation inhibitor step, in which a label degradation inhibitor is deposited onto the surface of the sample exposed array, i.e., an array that has been previously contacted with a fluorescently labeled sample. In this label degradation inhibitor deposition step, the ligand displaying surface of the array is contacted with a low surface tension deposition fluid that includes a fluorescent label degradation inhibitor, such that the degradation inhibitor is deposited onto the surface of the array, and specifically in the features of the array, as described in greater detail below. This label degradation inhibitor deposition step provides a number of benefits, which benefits are reviewed in greater detail below.

As summarized above, the deposition fluid employed in the wash step is a low surface tension fluid. As such, the surface tension of the fluid employed in this wash step typically does not exceed about 40, and in certain embodiments does not exceed about 35, including about 30 mN/m (as measured at 25° C.). (The determination of a given fluid's surface tension is performed by well-known and standard procedures, and may also be made by referring to a reference source that provides the surface tension of various fluids at various temperatures).

In many embodiments,.the low surface tension deposition fluid is a low viscosity fluid. In such embodiments, the viscosity of the fluid typically does not typically exceed about 1.2, and in certain embodiments does not exceed about 0.6, such as about 0.4 cP (as measured at 25° C.). The non-dimensional capillary number of the flow should be in the range of from about 10⁻² to about 10⁻⁶ in certain embodiments. The capillary number Ca is defined as Ca=(μU)/σ, where μ is the viscosity, U is the linear speed and σ is the surface tension. This number provides a range within which the slide drag-out speed can be adjusted to account for the particular fluid properties. However, while Ca serves as a coarse guide for controlling mechanical aspects of the flow, other subtleties such as the evaporation rate and fluid adherence to the substrate manifested in the disjoining pressure influence the motion of the contact line.

In many embodiments, the low surface tension fluid is one that is miscible with the fluid that previously contacted the array surface in the particular protocol being performed, e.g., the sample or the previous wash fluid. As such, in many embodiments, the low surface tension fluid is one that is miscible with aqueous fluids. For purposes of the present invention, a first and second fluid are considered to be miscible if the first fluid is soluble in the second fluid when the two fluids are present in a ratio of first to second fluid of at least 0.25/1, such as at least about 0.5/1, including at least about 0.75/1, such as at least about 1/1.

In many embodiments, the low surface tension deposition fluid is one in which the analyte or ligands of the array, e.g., nucleic acids, is not soluble. In certain embodiments where the analyte and ligand therefore are nucleic acids, the low surface tension fluid is not a nucleic acid solvent, by which is meant that nucleic acids, e.g., DNA, RNA, as well as mimetics thereof, are not soluble in the low surface tension fluid. In these embodiments, the solubility of nucleic acids in the fluid is described as the fraction of hybridized nucleic acid that are melted upon contact with the fluid (as measured at Standard Temperature and Pressure). This fraction does not exceed about 20%, (including about 15%, about 10%, about 5%) and typically does not exceed about 1%, e.g., over a given time period, such as a period of at least about 10 min, including at least about 60 min, including at least about 6 hours or longer. In many embodiments, the low surface tension fluid used to deposit the label degradation inhibitor is further characterized-in that it is an organic solvent. (By organic solvent is meant a fluid made up of carbon containing molecules). Specific organic solvents of interest include, but are not limited to: acetonitrile, acetone, methanol, ethanol and the like.

In certain embodiments, the low surface tension fluid is one that does not include a cosolvent. In yet other embodiments, this fluid may include a cosolvent. When a cosolvent is present, the amount of the cosolvent typically will not exceed about 50% (v/v), such as about 20% (v/v). Representative cosolvents that may be present include, but are not limited to: acetonitrile, acetone, ethyal acetate, hexane, diethyl ether, methanol, ethanol, acetylacetone, diethylcarbonate, chloroform, methylene chloride, and the like.

The above described low surface tension fluid is used to place or deposit a fluorescent label (i.e., dye) degradation inhibitor onto the surface of the array, and particularly onto the features of the array. By fluorescent label degradation inhibitor is meant an agent that at least reduces or slows the degradation of fluorescent signal from a label over a given period of time, e.g., at least about 5 mins,, including at least about 48 hours, where the magnitude of reduction in degradation as compared to a control is at sometimes at least about 10-fold, including at least about 50-fold.

In many embodiments, the fluorescent label degradation inhibitor is an ozone mediated degradation inhibitor, but which is meant that it is an agent or compound that inhibits the label degradation activity of ozone. In other words, the degradation inhibitor is one that protects the fluorescent label from degradation caused by ozone. In many embodiments, the ozone mediated degradation inhibitor is an ozone scavenger or a scavenger of the reactive species formed from the reaction of ozone with other molecules. In certain embodiments, the agent employed is one that protects the fluorescent label from ozone mediated degradation both during and after drying of the array surface.

In many embodiments, the agent employed is one that has substantially little, if any, impact on the quantum yield of the fluorescent label of interest, (i.e., it does not quench the label) where a given agent has substantially little impact on the quantum yield of a fluorescent label if the magnitude of reduction in quantum yield when the agent is present as compared to when the agent is absent does not exceed about 10%, such as 2%. (As determined by evaluating a change in fluorescence intensity in the presence and absence of the agent under otherwise identical conditions). Furthermore, agents of interest in certain embodiments do not affect the binding member complexes on the surface of an array, e.g., do not affect hybridized nucleic acid structures on the surface of the array, and specifically do not disrupt binding member complexes, e.g., nucleic acid duplex structures, on the surface of the array.

Often, ozone scavengers of interest are organic compounds that are soluble in organic fluids, but substantially insoluble, if not completely insoluble, in water and aqueous fluids. For purposes of the present application, a compound is considered to be substantially insoluble in water if its solubility in water (as measured at Standard Temperature and Pressure) does not exceed about 1.0 μM, and for example does not exceed about 0.1 μM. A variety of different types of ozone scavengers may be employed.

One class of representative ozone scavengers that may be employed are phenols antioxidants, such as hindered phenols, for example Pentaerythritol tetrakis(3,5-di-tert-butyl-4-hydroxyhydrocinnamate)2,6-Di-tert-butyl-4-methylphenol, Butylated hydroxyanisole, 2,4-Di-tert-butylphenol; biphenyldiols, for example 3,3′,5,5′-Tetramethylbiphenyl-4,4′-diol; thiobisphenols; alkylidenebisphenols, for example 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)-phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis-(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methyl-phenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis-(3,5-di-tert-butyl-4-hydroxyphenyl)propane, 2,2-bis-(5-tert-butyl-4-hydroxy2-methylphenyl)-4-n-dodecylmercaptobutane, 1,1,5,5-tetra-(5-tert-butyl-4-hydroxy-2-methylphenyl)pentane, etc.; 5,5′-Methylenebis(2-hydroxy-4-methoxybenzophenone), etc. Alkyl Gallates, for example methyl gallate, ethyl gallate, propyl gallate, butyl gallate, Lauryl gallate, etc.; and

Another class of ozone scavengers of interest is the HALS family (hindered amine light stabilizers) of low and high molecular weights, where representative species of interest include, but are not limited to: 2,2,5,5-Tetramethyl-3-pyrrolidinecarboxamide; 1,5,8,12-Tetrakis[4,6-bis(N-butyl-N-1,2,2,6,6-pentamethyl-4-piperidylamino)-1,3,5-triazin-2-yl]-1,5,8,12-tetraazadodecane, 2,2,6,6-Tetramethyl-4-piperidinol, 1-HYDROXY-PIPERIDINE-2,6-DIONE, Hexahydro-2,6-bis(2,2,6,6-tetramethyl-4-piperidinyl)-1H,4H,5H,8H-2,3a,4a,6,7a,8a-hexaazacyclopenta[def]fluorene-4,8-dione, (1-HYDROXY-2,2,6,6-TETRAMETHYL-PIPERIDIN-4-YL)-UREA, 2,2,6,6-Tetramethylpiperidine, 2,2,5,5-Tetramethyl-3-pyrrolidinecarboxamide, 2,2,6,6-Tetramethyl-1-(1-phenylethoxy)piperidine, 2,2,6,6-Tetramethyl-4-piperidinol, HALS stabilizers sold under the tradmarks Fiberstab L 112®, Tinuvin 123 S®, Tinuvin 765®, Tinuvin 770 DF®, Tinuvin 783 FDL® (all of which are available from Ciba Specialty Chemicals); and the like; Radical scavengers from the TEMPO family and the like, such as 4-Oxo-TEMPO, 4-Phosphonooxy-TEMPO hydrate, free radical, 2,2,6,6-Tetramethylpiperidine 1-oxyl, 4-Methoxy-TEMPO; Radical scavengers from the carotene and retinal family and the like, such as trans-β-Carotene, Lutein, Lycopene, all-trans-Retinol, 1,3-cis-Retinal, Retinyl acetate and the like; the Triazine trione family and the like, such as 1,3,5-Triallyl-1,3,5-triazine-2,4,6(1H,3H,5H)-trione, 1,3,5-Tris(2-hydroxyethyl)-1,3,5-triazine-2,4,6(1H,3H,5H)-trione; Radical scavengers, for example Anthrone; other natural and synthetic antioxidants; mixture thereof; water soluble dispersions of an ozone scavenger, such as antioxidant dispersions in latex colloids;and the like.

The amount of label degradation inhibitor (e.g., ozone scavenger) that is present in the low surface tension in this deposition step of the subject methods may vary depending on the nature of the agent, but in many embodiments ranges from about 0.1 μM to about 250 μM, including from about 10 μM to about 100 μM .

In performing the label degradation inhibitor deposition step of the present invention in which the label degradation inhibitor is applied to the array surface, it may, in certain embodiments, be desirable to precede this deposition step with a wash step that is specifically designed to remove unbound components from the array surface that are insoluble in the low surface tension fluid employed in the degradation inhibitor deposition step. For example, a given array assay protocol may include a wash step in which a wash fluid that includes agents, e.g., surfactants, that are insoluble in the low surface tension fluid employed in the deposition step. In such embodiments, it may be desirable to include a wash step, e.g., with a solvent for the surfactant (such as n-propyl alcohol) that removes these agents from the array surface prior to performing the inhibitor deposition step.

The inhibitor deposition step may be performed using any convenient protocol. In many embodiments, this deposition step includes immersing the array in a sufficient volume of a low surface tension fluid that includes the degradation inhibitor and then removing the array from the fluid. While immersed, the array and/or fluid may be agitated as desired. In certain embodiments, the array may be removed from the fluid at a constant rate, e.g., at a rate of from about 0.1 cm/sec to about 10 cm/sec.

Because of the nature of the low surface tension fluid as described above, the array surface contacted with the fluid is essentially dry immediately upon removal of the array surface from the fluid. Accordingly, no separate drying step is needed following contact of the array surface with the low surface tension fluid. In certain embodiments, contact with the low surface tension may be viewed as a surface drying step.

In certain embodiments, it may be desirable to include an additional drying step, such as gas, e.g., air or nitrogen, knife drying; centrifuge drying; squeegee drying; evaporation; etc.

The degradation inhibitor deposition step, as described above, may be incorporated into an automated array processing, e.g., assaying protocol, in which one or more of the individual steps of the protocol, including the deposition step, are performed using automated machinery or instruments.

Arrays processed according to the subject methods that include a degradation inhibitor deposition step have unique properties that distinguish them from arrays processed by other methods in which the subject label degradation inhibitor deposition step of the present invention is not employed. In certain embodiments, the arrays processed by the subject methods are ones having an amount of a label degradation inhibitor in one or more, and typically of all, the features of the array. In many embodiments, because the nature of the deposition step, a uniform coating of the label degradation inhibitor is present in each feature of the array. In such embodiments, the uniform coating of degradation inhibitor in each feature may vary in thickness, but may range in thickness from about 1 molecular layer to about 10 μm, including from about 1 molecular layer to about 0.1 μm. In certain embodiments, e.g., where the array is an in situ produced nucleic acid array that is characterized by having features with surface engery and interfeature regions of low surface energy, the degradation inhibitor is found only in each feature, with substantially little if any feature modification agent present in interfeature areas.

Following the degradation inhibitor deposition step, and any desired storage period, the presence of any resultant binding complexes on the array surface is then detected, e.g., through use of a signal production system, a fluorescent label signal production system. In other words, the resultant dried array is then interrogated or read to detect the presence of any binding complexes on the surface thereof, e.g., the label is detected using fluorimetric detection protocols means. The presence of the analyte in the sample is then deduced or determined from the detection of binding complexes on the substrate surface.

Utility

The methods of the present invention find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., a fluorescent label present on the analyte, etc. The presence of the analyte in the sample is then deduced from the detection of binding complexes on the substrate surface.

Specific analyte detection applications of interest include hybridization assays in which the nucleic acid arrays of the invention are employed. In these assays, a sample of target nucleic acids is first prepared, where preparation may include labeling of the target nucleic acids with a fluorescent label. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest that may be practiced using the arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Where the arrays are arrays of polypeptide binding agents, e.g., protein arrays, specific applications of interest include analyte detection/proteomics applications, including those described in: U.S. Pat. Nos. 4,591,570; 5,171,695; 5,436,170; 5,486,452; 5,532,128; and 6,197,599; the disclosures of which are herein incorporated by reference; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

In certain embodiments, the methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., nucleic acid or protein containing sample) and the array then read, following the subject wash in a low surface tension fluid that includes the label degradation inhibitor. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER scanner available from Agilent Technologies, Palo Alto, Calif.. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or whether an organism from which the sample was obtained exhibits a particular condition, for example, cancer). The results of the reading (processed or not) may be forwarded (such as by communication) to a remote location if desired, and received there for further use (such as further processing).

Kits

Kits for use in analyte detection assays, as described above, are also provided. The kits at least include a low surface tension deposition fluid and a label degradation inhibitor, as described above, where these two components may or may not be combined into a single composition. The kits may further include one or more additional components necessary for carrying out an analyte detection assay, such as one or more ligand arrays, sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include buffers (such as hybridization buffers), wash mediums, enzyme substrates, reagents for generating a labeled target sample such as a labeled target nucleic acid sample, negative and positive controls and written instructions for using the array assay devices for carrying out an array based assay.

Such kits also typically include instructions for use in practicing array-based assays according to the subject invention. The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The following examples are offered by way of illustration and not by way of limitation.

Experimental

I. Atmospheric Ozone Causes Cy5 Signal Degradation

A. Materials and Methods

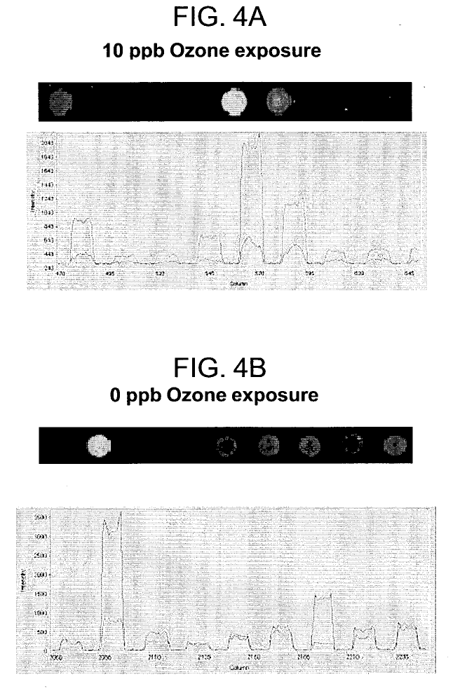

An Agilent in situ Human catalog array (part #G4110A) was hybridized to a sample of 1.5 μg Cy3/Cy5 labeled RNA (Cy3 channel was MG63 cell line and Cy5 channel was brain) and washed using the protocols described in Hughes et al., Expression Profiling using microarrays fabricated by an ink-jet oligonucleotide synthesizer, Nature, 2001, 19, 342, in a controlled atmosphere of 10 ppb of ozone. Per the protocol, at the end of the 2^{nd} wash, the array was removed from the 0.06×SSPE buffer and a droplet of buffer was present on every feature. Upon drying of every droplets (20 to 30 sec), the array was removed from the ozone filled atmosphere and stored in an ozone free atmosphere until scanning. Control slides were processed in parallel and the final drying was performed in an ozone free environment. FIGS. 4A and 4B show the morphologies of representative features from this experiment (A, 10 ppb ozone concentration; B, 0 ppb ozone concentration). Individual features of slides dried in a 10 ppb ozone environment are characterized by a non-uniform log ratio (green/red signal), a normal green signal profile and conical red profile signal. On the contrary, slides processed in a 0 ppb ozone environment are characterized by a uniform log ratio (green/red signal), a normal green signal profile and normal red profile signal. This demonstrates a non-uniform degradation of the Cy5 dye by ozone within individual features.

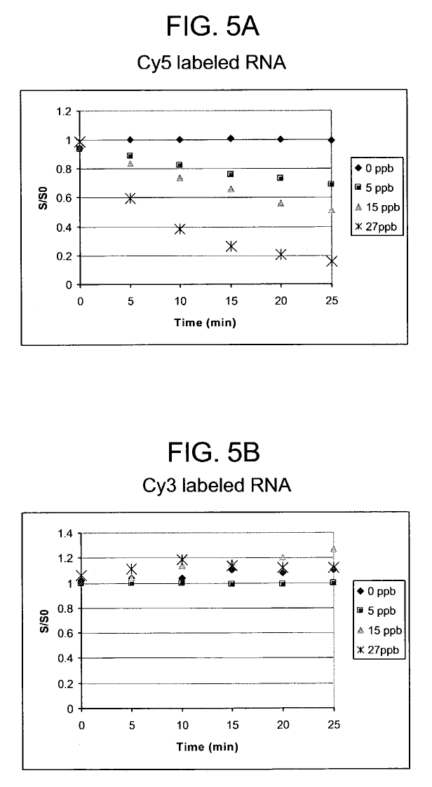

An Agilent in situ Human catalog array (part #G4110A) was hybridized to a sample of 1.5 μg Cy3/Cy5 labeled RNA (Cy3 channel was MG63 cell line and Cy5 channel was brain) and washed using the protocols described in Hughes et al., Expression Profiling using microarrays fabricated by an ink-jet oligonucleotide synthesizer, Nature, 2001, 19, 342, in a controlled atmosphere free of ozone. Per the protocol, at the end of the 2nd wash, the array was removed from the 0.06×SSPE buffer and a droplet of buffer was present on every feature. Upon drying of every droplets (10 to 20 sec), the array was scanned then placed in a controlled atmosphere of 0 ppb of ozone for 5 min, then scanned again. The ozone exposure and scanning steps were repeated until the total array exposure reached 30 min. The images were analyzed and the plot of the ratio of signal after each exposure over the initial signal is shown on figure FIGS. 5A and B. The same experiment was repeated with an ozone concentration of 5, 15 and 27 ppb. FIGS. 5A and 5B show that the degradation of Cy5 is a pseudo first order reaction correlated with the concentration of ozone. FIGS. 5A and 5B also show that Cy3 is not significantly affected by ozone at the concentration tested. Analysis of the feature morphologies indicates that the signal loss is uniform within individual features (not shown).

B. Results and Discussion

The above results demonstrate that the degradation of the Cy dye fluorescent signal primarily occurs after the hybridization and washing steps, which leads to erroneous log ratios and/or non-uniform signal distributions within features. Furthermore, the above results demonstrate that this degradation is due to the action of ozone gas present in the atmosphere. The source of ozone has been correlated to the action of the sun on the atmospheric pollution, resulting in a diurnal variation in the observed ambient ozone concentration. The chemical mechanism by which ozone reacts with Cy dyes is unknown, and could be by direct oxidation, or free radical reaction, such as with peroxide radicals generated from ozone.

Furthermore, the above demonstrates that there are several ozone degradation modes:

The first is a rapid degradation during the drying step of array processing. It is characterized by usual signal profile of individual features in the green channel (step function or batman shape) but unusual signal profile in the red channel (conical shape). Therefore, non-uniform log ratios are observed for pixels of individual features. The conical shapes of the red line profiles indicate a connection between the Cy5 dye degradation and the drying of individual feature droplets after the slide has been removed from the wash solution. During the same time, the Cy3 dye is not affected. This failure mode has been observed at ozone level down to 10 ppb when no surfactant is used in the last wash buffer. The reaction time for this mode is less than a few seconds.

The second mode is a slower reaction after the slide surface has been dried. This typically occurs after drying while the slide is placed in the scan holder (and the carousel) before scanning. It is characterized by a continuous decrease in red signal intensity from the left side to the right side of the array when the slide is in the scan holder or a uniform drop in red signal intensity if the slide is not in a confined space. Within individual feature, the decrease in signal intensity is always uniform. This mode has been observed after-exposures on the order of minutes to tens of minutes at ozone concentration above 5 ppb.

II. Use of an Ozone Scavenger Eliminates the Degradation Effect of Atmospheric Ozone on Cy5

A. Materials and Methods

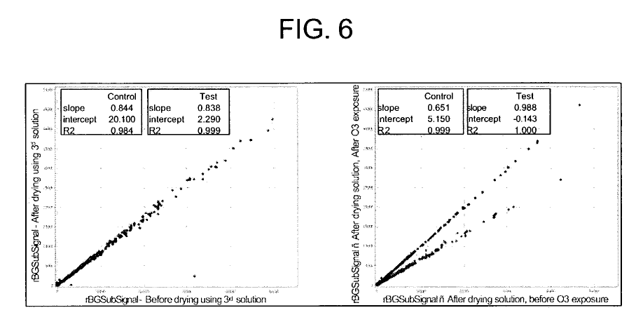

Ozone scavenger screening: An Agilent in situ Human catalog array (part #G4110A) was hybridized to a sample of 1.5 μg Cy3/Cy5 labeled RNA (Cy3 channel was MG63 cell line and Cy5 channel was brain) and washed using the current recommended protocols, as described in Agilent Publication Number G4140-90010. Per the protocol, at the end of the 2^{nd} wash, the array was coated with a sheet of 0.06×SSC buffer and 0.05% Triton-X 102 as surfactant. The array was then dried and scanned. Then, half of the array was transferred to a 3^{rd} wash solution of 50 mM of 2,2′-Methylenebis(6-tert-butyl-4-methylphenol) in acetonitrile. After 30 seconds, the array was removed from the solution at a constant speed. This action resulted in the array being completely dried. The array was scanned, exposed to an atmosphere of 50 ppb of ozone for 5 min, then scanned again. Results are shown in FIG. 6.

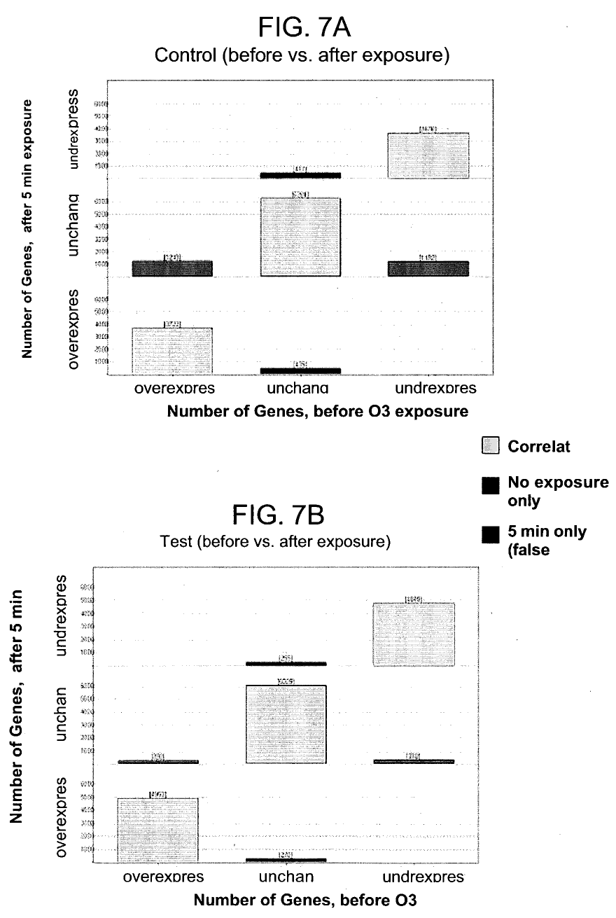

Scavenger validation: Eight Agilent in situ Human catalog arrays (part #G4110A) were hybridized to a sample of 1.5 μg Cy3/Cy5 labeled RNA (MG63/brain) in a dye swap experiment (4 of polarity 1 and 4 of polarity −1). 4 arrays (controls) were washed using the current recommended protocols, as described in Agilent Publication Number G4140-90010. Per the protocol, at the end of the 2^{nd} wash, the arrays were coated with a sheet of 0.06×SSC buffer and 0.05% Triton-X 102 as surfactant, and they were then dried and scanned. 4 arrays (test) were washed using the current recommended protocols, as described in Agilent Publication Number G4140-90010. Per the protocol, at the end of the 2^{nd} wash, the arrays were coated with a sheet of 0.06×SSC buffer and 0.05% Triton-X 102 as surfactant. Instead of performing the standard drying process following these washes, the array was then transferred to a 3^{rd} wash solution of 25 mM Pentaerythritol tetrakis(3,5-di-tert-butyl-4-hydroxyhydrocinnamate) in acetonitrile. Optionally, the arrays may be transferred from wash 2 into a container containing n-propyl alcohol prior to transfer into the acetonitrile solution. This will remove the surfactant and prevent its precipitation in acetonitrile. Alternatively, no surfactant or a different buffer formulation may be used in wash 2. An example of such as formulation is 0.06×SSPE at room temperature. The array were then scanned. After scanning, all eight arrays were exposure to 50 ppb of ozone for 5 min and re-scanned. The arrays were analyzed and the gene expression significance of each probes was calculated at 95% confidence based on the log ratio×polarity of both dye swaps being different from zero at 95% confidence. In other words, probes were called differentially expressed if, at 95% confidence, the range centered at the log ratio×polarity and having a width equal to two standard deviations did not cross zero. The numbers of genes found differentially expressed in each treatment, i.e. control and no exposure, control and 5 min exposure, test and no exposure, test and 5 min exposure were calculated and compared. Results are shown in figures FIG. 7A and 7B.

B. Results and Discussion

The results of the above screening assay are provided in Figure New FIG. 6. FIG. 6 shows the effect of the drying solution on signal (left) and protection against ozone exposure by the reagent dissolved in the drying solution (right). The solution was 50 mM of 2,2′-Methylenebis(6-tert-butyl-4-methylphenol) in Acetonitrile. The “control” features (red) were not dried in the 3^{rd} solution while the “test” features were. There is no effect of the drying solution on the signal intensity (left, similar slopes). The Cy5 signals of control features are decreased by 45% (right, slope of 0.65) upon 5 min ozone exposure at 50 ppb, while the signals of “test” features protected by the ozone scavenger only decrease by 1.2% (slope of 0.988).

The results of the validation assay are provided in FIGS. 7A and 7B, which shows the effect of the drying solution on the number of genes found differentially expressed and on the protection against ozone exposure by the reagent dissolved in the drying solution. The solution was 25 mM of Pentaerythritol tetrakis(3,5-di-tert-butyl-4-hydroxyhydrocinnamate) in Acetonitrile. FIG. 7A shows that the number of false positive and false negative in the control arrays i.e. without wash in the 3^{rd} solution, are significantly increased after ozone exposure compared to test arrays (FIG. 7B), i.e. with wash in the 3rd solution.

It is evident from the above results and discussion that the above-described invention provides a greatly improved method of performing array-based assays. Specifically, the subject invention provides for effective inhibition of ozone mediated degradation of fluorescent labels on the surface of the array, both during and after drying, leading to significantly improved results in array-based assays that are performed under environmental conditions that include ozone. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of determining whether a fluorescently labeled analyte is present in a sample, said method comprising:
(a) exposing a surface of a substrate having immobilized thereon a ligand that specifically binds to said fluorescently labeled analyte to said sample;
(b) contacting said surface with a low surface tension fluid degradation inhibitor deposition fluid that comprises a fluorescent label degradation inhibitor; and
(c) detecting any resultant binding complexes on said surface to determine whether said analyte is present in said sample.

2. The method according to claim 1, wherein said deposition fluid has a surface tension that does not exceed about 40 mN/m.

3. The method according to claim 1, wherein said deposition fluid is a low viscosity fluid.

4. The method according to claim 1, wherein said deposition fluid has a viscosity that does not exceed about 1.2 cP.

5. The method according to claim 1, wherein said deposition fluid is an organic fluid.

6. The method according to claim 5, wherein said deposition fluid comprises acetonitrile.

7. The method according to claim 1, wherein said fluorescent dye degradation inhibitor is an ozone mediated degradation inhibitor.

8. The method according to claim 7, wherein said ozone mediated degradation inhibitor is an ozone scavenger.

9. The method according to claim 1, wherein said analyte is a nucleic acid.

10. The method according to claim 1, wherein said ligand is a nucleic acid.

11. The method according to claim 10, wherein said substrate displaying said nucleic acid ligand is a nucleic acid array.

12. The method according to claim 11, wherein said nucleic acid array is an in situ prepared nucleic acid array.

13. The method according to claim 1, wherein said method further comprises at least one wash step prior to said inhibitor deposition step (b).

14. The method according to claim 13, wherein said at least one wash step comprises washing said substrate surface with an aqueous fluid.

15. The method according to claim 1, wherein said method is a method of assaying said sample for the presence of two or more distinct analytes.

16. A method comprising transmitting data representing a result obtained by the method according to claim 1, from a first location to a second location.

17. A method according to claim 16, wherein said second location is a remote location.

18. A method comprising receiving data representing a result of a method of claim 1.

19. A kit for performing an assay, said kit comprising:
(a) a low surface tension degradation inhibitor deposition fluid; and
(b) a fluorescent label degradation inhibitor.

20. The kit according to claim 19, wherein said deposition fluid comprises said degradation inhibitor.

21. The kit according to claim 20, wherein said deposition fluid and degradation inhibitor are present as separation compositions.

22. The kit according to claim 19, wherein said kit further comprises an array.

23. The kit according to claim 19, wherein said deposition fluid has a surface tension that does not exceed about 40 mN/m.

24. The kit according to claim 19, wherein said deposition fluid is a low viscosity fluid.

25. The kit according to claim 24, wherein said deposition fluid has a viscosity that does not exceed about 1.2 cP.

26. The kit according to claim 19, wherein said deposition fluid is an organic fluid.

27. The kit according to claim 26, wherein said deposition fluid comprises acetonitrile.

28. The kit according to claim 19, wherein said kit further comprises an array comprising at least two different ligands.

29. The kit according to claim 28, wherein said ligands are nucleic acid ligands.

30. An array processed according to the method of claim 1.

31. The array according to claim 30, wherein said array is a nucleic acid array.

32. The array according to claim 31, wherein said nucleic acid array is an in situ prepared nucleic acid array.
